(19) United States
(12) Patent Application Publication
(10) Pub. No.: US 2018/0135085 A1
(43) Pub. Date: May 17, 2018

(54) AMINO ACID PRODUCTION

(71) Applicant: EVONIK DEGUSSA GMBH, Essen (DE)

(72) Inventors: Thomas HAAS, Münster (DE); Simon BECK, Münster (DE); Thomas BÜLTER, Duisburg (DE); Anja HECKER née THIESSENHUSEN, Münster (DE); Martin DEMLER, Dorsten (DE)

(73) Assignee: EVONIK DEGUSSA GMBH, Essen (DE)

(21) Appl. No.: 15/574,334

(22) PCT Filed: Jun. 21, 2016
(86) PCT No.: PCT/EP2016/064265

§ 371 Date: Nov. 15, 2017

(30) Foreign Application Priority Data
Jul. 10, 2015 (EP) 15176246.5
Dec. 17, 2015 (EP) 15200706.8

(51) Int. Cl. C12P 13/06; C12P 7/54

(52) U.S. Cl. CPC C12P 13/06; C12P 7/54

(57) ABSTRACT

The present invention relates to a method of producing at least one amino acid from a carbon source in aerobic conditions, the method comprising: (a)step of producing ethanol and/or acetate from the carbon source in aerobic conditions, comprising (i)contacting a reaction mixture comprising—a first acetogenic microorganism in an exponential growth phase; —free oxygen; and —a second acetogenic microorganism in a stationary phase wherein the first and second acetogenic microorganism is capable of converting the carbon source to the acetate and/or ethanol; and (b)step of contacting the acetate and/or ethanol from step (a) with a third microorganism capable of converting the acetate and/or ethanol to at least one amino acid.

### FIELD OF THE INVENTION

The present invention relates to a biotechnological method for producing amino acids. In particular, the method may use carbon monoxide and/or carbon dioxide as the starting material.

### BACKGROUND OF THE INVENTION

Amino acids are especially useful as additives in animal feed and as nutritional supplements for human beings. They can also be used in infusion solutions and may function as synthetic intermediates for the manufacture of pharmaceuticals and agricultural chemicals. Compounds such as cysteine, homocysteine, methionine and S-adenosylmethionine are usually industrially produced to be used as food or feed additives and also in pharmaceuticals. In particular, methionine, an essential amino acid, which cannot be synthesized by animals, plays an important role in many body functions. D,L-methionine is presently being produced by chemical synthesis from hydrogen cyanide, acrolein and methyl mercaptan. These petroleum based starting materials such as acrolein and methyl mercaptan are obtained by cracking gasoline or petroleum which is bad for the environment. Also, since the costs for these starting materials will be linked to the price of petroleum, with the expected increase in petroleum prices in the future, prices of methionine will also increase relative to the increase in the petroleum prices.

There are several chemical means of producing methionine. In one example, 3-methylthiopropanal is used as a raw material with hydrocyanic acid in the presence of a base. The reaction results in ammonium carbonate, which is then hydrolysed. In this method, carbon dioxide is introduced into the reaction liquid after hydrolysis, whereby crystallization occurs and methionine is separated as a crystal. Carbon dioxide and hydrogen are used as raw materials for producing methionine using this method. However, a large amount of hydrogen is left over, making this method inefficient.

With the increasing methionine demand, thus microbial production of methionine is always an attractive alternative.

The pathway for L-methionine synthesis is well known in many microorganisms (Figge R M (2006)). E. coli and C. glutamicum methionine producer strains have also been described in patent applications WO2005/111202, WO2007/077041, WO2007/012078 and WO2007/135188. Methionine produced by fermentation needs to be purified from the fermentation broth. Cost-efficient purification of methionine relies on producer strains and production processes that minimize the amount of by-products in the fermentation broth. Further, most of these biotechnological methods of producing methionine use nutrients including, but not limited to, carbohydrate sources, e.g., sugars, such as glucose, fructose, or sucrose, hydrolyzed starch, nitrogen sources, e.g., ammonia, and sulfur sources e.g., sulfate and/or thiosulfate, together with other necessary or supplemental media components as a starting material. This process would yield L-methionine and biomass as a by-product with no toxic dangerous, flammable, unstable, noxious starting materials. However, this is an expensive raw material and the yield too low to consider this method commercially viable.

Accordingly, there is a need in the art for a cheaper and more efficient biotechnological means of producing methionine.

### DESCRIPTION OF THE INVENTION

The present invention provides a biotechnological means of producing at least one amino acid from a carbon source in aerobic conditions. The carbon source may comprise carbon dioxide and/or carbon monoxide. In particular, the method comprises at least two parts. One part that involves the formation of acetate and/or ethanol from a carbon source and a further part which involves the use of the acetate and/or ethanol in the formation of at least one amino acid.

In one aspect of the present invention, there is provided a method of producing at least one amino acid from a carbon source in aerobic conditions, the method comprising:

(a) a step of producing ethanol and/or acetate from the carbon source in aerobic conditions, comprising
(i) contacting a reaction mixture comprising
   a first acetogenic microorganism in an exponential growth phase;
   free oxygen; and
   a second acetogenic microorganism in a stationary phase

wherein the first and second acetogenic microorganism is capable of converting the carbon source to the acetate and/or ethanol; and

(b) a step of contacting the acetate and/or ethanol from step (a) with a third microorganism capable of converting the acetate and/or ethanol to the amino acid.

A microorganism capable of converting acetate and/or ethanol to the amino acid may refer to any microorganism that may be able to carry out fermentative production of amino acids. In particular for producing L-amino acids. The term “L-amino acid-producing microorganism” refers to microorganisms which, when contacted with a substrate, convert the substrate to an L-amino acid. These microorganisms may produce the appropriate enzymes intracellularly and/or extracellularly. These amino acid-producing microorganisms may be capable of utilising starting material for amino acid fermentation that may be waste materials. For instance, syngas and the ethanol and/or acetate derived from syngas may be utilized for the amino acid production. This is particularly advantageous as inexpensive starting materials can be utilized that would originally have been considered waste. This also enables the removal of waste which consequently reduces environmental pollution.

According to any aspect of the present invention, the amino acid may be selected from the group consisting of L-alanine, L-glycine, L-glutamate, L-lysine, L-homoserine, L-isoleucine, L-threonine and acetyl homoserine. In particular, the amino acid may be L-lysine, L-homoserine and acetyl homoserine. More in particular, the amino acid produced by the method according to any aspect of the present invention may be L-homoserine. Even more in particular, the amino acid produced by the method according to any aspect of the present invention may be acetyl homoserine. In one example, the amino acid produced according to any aspect of the present invention may be L-homoserine and/or acetyl homoserine. In particular, the amino acid may be I acetyl homoserine. More in particular, the third microorganism may be genetically modified to comprise increased expression relative to the wild type cell of homoserine acetyl transferase (E₁), aspartokinase (E₂) and homoserine dehydrogenase (E₃); and at least one enzyme selected from a group consisting of phosphoenolpyruvate carboxylase (E₄), aspartate aminotransferase (E₅) and aspartate semi-aldehyde dehydrogenase (E₆).

The term “acetate” as used herein, refers to both acetic acid and salts thereof, which results inevitably, because as known in the art, since the microorganisms work in an aqueous environment, and there is always a balance between salt and acid present.

In particular, the second acetogenic microorganism in a post exponential phase may be in the stationary phase of the cell. The acetogenic cells in the log phase allow for any other acetogenic cells in the aqueous medium to produce acetate and/or ethanol in the presence of oxygen. The concentration of acetogenic cells in the log phase may be maintained in the reaction mixture. Therefore, at any point in time in the reaction, the reaction mixture comprises acetogenic cells in the log phase and acetogenic cells in another growth phase, for example in the stationary phase.

A skilled person would understand the different growth phases of microorganisms and the methods to measure them and identify them. In particular, most microorganisms in batch culture, may be found in at least four different growth phases; namely they are: lag phase (A), log phase or exponential phase (B), stationary phase (C), and death phase (D). The log phase may be further divided into the early log phase and mid to late log/exponential phase. The stationary phase may also be further distinguished into the early stationary phase and the stationary phase. For example, Cotter, J. L., 2009, Najafpour. G., 2006, Younesi, H., 2005, and Köpke, M., 2009 disclose different growth phases of acetogenic bacteria. In particular, the growth phase of cells may be measured using methods taught at least in Shuler M L, 1992 and Fuchs G., 2007.

The lag phase is the phase immediately after inoculation of the cells into a fresh medium, the population remains temporarily unchanged. Although there is no apparent cell division occurring, the cells may be growing in volume or mass, synthesizing enzymes, proteins, RNA, etc., and increasing in metabolic activity. The length of the lag phase may be dependent on a wide variety of factors including the size of the inoculum; time necessary to recover from physical damage or shock in the transfer; time required for synthesis of essential coenzymes or division factors; and time required for synthesis of new (inducible) enzymes that are necessary to metabolize the substrates present in the medium.

The exponential (log) phase of growth is a pattern of balanced growth wherein all the cells are dividing regularly by binary fission, and are growing by geometric progression. The cells divide at a constant rate depending upon the composition of the growth medium and the conditions of incubation. The rate of exponential growth of a bacterial culture is expressed as generation time, also the doubling time of the bacterial population. Generation time (G) is defined as the time (t) per generation (n=number of generations). Hence, G=t/n is the equation from which calculations of generation time derive. The exponential phase may be divided into the (i) early log phase and (ii) mid to late log/exponential phase. A skilled person may easily identify when a microorganism, particularly an acetogenic bacteria, enters the log phase. For example, the method of calculating the growth rate of acetogenic bacteria to determine if they are in the log phase mey be done using the method taught at least in Henstra A. M., 2007. In particular, the microorganism in the exponential growth phase according to any aspect of the present invention may include cells in the early log phase and mid to late log/exponential phase.

The stationary phase is the phase where exponential growth ends as exponential growth cannot be continued forever in a batch culture (e.g. a closed system such as a test tube or flask). Population growth is limited by one of three factors: 1. exhaustion of available nutrients; 2. accumulation of inhibitory metabolites or end products; 3. exhaustion of space, in this case called a lack of “biological space”. During the stationary phase, if viable cells are being counted, it cannot be determined whether some cells are dying and an equal number of cells are dividing, or the population of cells has simply stopped growing and dividing. The stationary phase, like the lag phase, is not necessarily a period of quiescence. Bacteria that produce secondary metabolites, such as antibiotics, do so during the stationary phase of the growth cycle (Secondary metabolites are defined as metabolites produced after the active stage of growth).

The death phase follows the stationary phase. During the death phase, the number of viable cells decreases geometrically (exponentially), essentially the reverse of growth during the log phase.

In one example, where O₂ is present in the reaction mixture according to any aspect of the present invention, the first acetogenic bacteria may be in an exponential growth phase and the other acetogenic bacteria may be in any other growth phase in the lifecycle of an acetogenic microorganism. In particular, according to any aspect of the present invention, the acetogenic bacteria in the reaction mixture may comprise one acetogenic bacteria in an exponential growth phase and another in the stationary phase. In the presence of oxygen, without the presence of the acetogenic bacteria in an exponential growth, the acetogenic bacteria in the stationary phase may not be capable of producing acetate and/or ethanol. This phenomenon is confirmed at least by Brioukhanov, 2006, Imlay, 2006, Lan, 2013 and the like. The inventors thus surprisingly found that in the presence of acetogenic bacteria in an exponential growth, the acetogenic bacteria in any growth phase may aerobically respire and produce acetate and/or ethanol at more than or equal to the amounts produced when the reaction mixture was absent of oxygen. In one example, the acetogenic bacteria in the exponential growth phase may be capable of removing the free oxygen from the reaction mixture, providing a suitable environment (with no free oxygen) for the acetogenic bacteria in any growth phase to metabolise the carbon substrate to produce acetate and/or ethanol.

In another example, the aqueous medium may already comprise acetogenic bacteria in any growth phase, particularly in the stationary phase, in the presence of a carbon source. In this example, there may be oxygen present in the carbon source supplied to the aqueous medium or in the aqueous medium itself. In the presence of oxygen, the acetogenic bacteria may be inactive and not produce acetate and/or ethanol prior to the addition of the acetogenic bacteria in the exponential growth phase. In this very example, the acetogenic bacteria in the exponential growth phase may be added to the aqueous medium. The inactive acetogenic bacteria already found in the aqueous medium may then be activated and may start producing acetate and/or ethanol.

In a further example, the acetogenic bacteria in any growth phase may be first mixed with the acetogenic bacteria in the exponential growth phase and then the carbon source and/or oxygen added.

According to any aspect of the present invention, a microorganism in the exponential growth phase grown in the presence of oxygen may result in the microorganism gaining an adaptation to grow and metabolise in the presence of oxygen. In particular, the microorganism may be capable of removing the oxygen from the environment surrounding the microorganism. This newly acquired adaptation allows for the acetogenic bacteria in the exponential growth phase to rid the environment of oxygen and therefore produce acetate and ethanol from the carbon source. In particular, the acetogenic bacteria with the newly acquired adaptation allows for the bacteria to convert the carbon source to acetate and/or ethanol.

In one example, the acetogenic bacteria in the reaction mixture according to any aspect of the present impression may comprise a combination of cells: cells in the log phase and cells in the stationary phase. In the method according to any aspect of the present invention the acetogenic cells in the log phase may comprise a growing rate selected from the group consisting of 0.01 to 2 h⁻¹, 0.01 to 1 h⁻¹, 0.05 to 1 h⁻¹,0.05 to 2 h⁻¹ 0.05 to 0.5 h⁻¹ and the like. In one example, the OD₆₀₀ of the cells of the log phase acetogenic cells in the reaction mixture may be selected from the range consisting of 0.001 to 2, 0.01 to 2, 0.1 to 1, 0.1 to 0.5 and the like. A skilled person would be able to use any method known in the art to measure the OD₆₀₀ and determine the growth rate of the cells in the reaction mixture and/or to be added in the reaction mixture. For example, Koch (1994) may be used. In particular, bacterial growth can be determined and monitored using different methods. One of the most common is a turbidity measurement, which relies upon the optical density (OD) of bacteria in suspension and uses a spectrophotometer. The OD may be measured at 600 nm using a UV spectrometer.

In order to maintain the concentration of the first and second acetogenic bacteria in the reaction mixture, a skilled person may be capable of extracting a sample at fixed time points to measure the OD₆₀₀, pH, concentration of oxygen and concentration of ethanol and/or higher alcohols formed. The skilled person would then be able to add the necessary component(s) to maintain the concentration of first and second acetogenic bacteria in the reaction mixture and to ensure an optimum environment is maintained for the production of ethanol and/or acetate.

The term “acetogenic bacteria” as used herein refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii O-52 (ATCC 55989), Clostridium mayombei (DSM 6539), Clostridium methoxybenzovorans (DSM 12182), Clostridium neopropionicum sp, Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui). More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ER12, Clostridium ljungdahlii COL and Clostridium ljungdahlii O-52 may be used in the conversion of synthesis gas to hexanoic acid. These strains for example are described in WO 98/00558, WO 00/68407, ATCC 49587, ATCC 55988 and ATCC 55989. The first and second acetogenic bacteria used according to any aspect of the present invention may be the same or different bacteria. For example, in one reaction mixture the first acetogenic bacteria may be Clostridium ljungdahlii in the log phase and the second acetogenic bacteria may be Clostridium ljungdahlii in the stationary phase. In another example, in the reaction mixture the first acetogenic bacteria may be Clostridium ljungdahlii in the log phase and the second acetogenic bacteria may be Clostridium carboxidivorans in the stationary phase.

In the reaction mixture according to any aspect of the present invention, there may be oxygen present. It is advantageous to incorporate O₂ in the reaction mixture and/or gas flow being supplied to the reaction mixture as most waste gases including synthesis gas comprises oxygen in small or large amounts. It is difficult and costly to remove this oxygen prior to using synthesis gas as a carbon source for production of higher alcohols. The method according to any aspect of the present invention allows the production of at least one higher alcohol without the need to first remove any trace of oxygen from the carbon source. This allows for time and money to be saved.

More in particular, the O₂ concentration in the gas flow may be may be present at less than 1% by volume of the total amount of gas in the gas flow. In particular, the oxygen may be present at a concentration range of 0.000005 to 2% by volume, at a range of 0.00005 to 2% by volume, 0.0005 to 2% by volume, 0.005 to 2% by volume, 0.05 to 2% by volume, 0.00005 to 1.5% by volume, 0.0005 to 1.5% by volume, 0.005 to 1.5% by volume, 0.05 to 1.5% by volume, 0.5 to 1.5% by volume, 0.00005 to 1% by volume, 0.0005 to 1% by volume, 0.005 to 1% by volume, 0.05 to 1% by volume, 0.5 to 1% by volume, 0.55 to 1% by volume, 0.60 to 1% by volume, particularly at a range of 0.60 to 1.5%, 0.65 to 1%, and 0.70 to 1% by volume in the gas phase of the gas flow and/or in the medium. In particular, the acetogenic microorganism is particularly suitable when the proportion of O₂ in the gas phase/flow is about 0.00005, 0.0005, 0.005, 0.05, 0.5, 0.6, 0.7, 0.8, 0.9, 1, 1.5, 2% by volume in relation to the volume of the gas in the gas flow. A skilled person would be able to use any one of the methods known in the art to measure the volume concentration of oxygen in the gas flow. In particular, the volume of oxygen may be measured using any method known in the art. In one example, a gas phase concentration of oxygen may be measured by a trace oxygen dipping probe from PreSens Precision Sensing GmbH. Oxygen concentration may be measured by fluorescence quenching, where the degree of quenching correlates to the partial pressure of oxygen in the gas phase. Even more in particular, the first and second microorganisms according to any aspect of the present invention are capable of working optimally in the aqueous medium when the oxygen is supplied by a gas flow with concentration of oxygen of less than 1% by volume of the total gas, in about 0.015% by volume of the total volume of gas in the gas flow supplied to the reaction mixture.

According to any aspect of the present invention, the aerobic conditions in which the carbon source is converted to ethanol and/or acetate in the reaction mixture refers to gas surrounding the reaction mixture. The gas may comprise at least 1% by volume of the total gas of oxygen and other gases including carbon sources such as CO, CO₂ and the like.

The aqueous medium according to any aspect of the present invention may comprise oxygen. The oxygen may be dissolved in the medium by any means known in the art. In particular, the oxygen may be present at 0.5mg/L in the absence of cells. In particular, the dissolved concentration of free oxygen in the aqueous medium may at least be 0.01mg/L. In another example, the dissolved oxygen may be about 0.01, 0.02, 0.03, 0.04, 0.05, 0.1, 0.2, 0.3, 0.4, 0.5 mg/L. In particular, the dissolved oxygen concentration may be 0.01-0.5mg/L, 0.01-0.4mg/L, 0.01-0.3mg/L, 0.01-0.1mg/L. In particular, the oxygen may be provided to the aqueous medium in a continuous gas flow. More in particular, the aqueous medium may comprise oxygen and a carbon source comprising CO and/or CO2. More in particular, the oxygen and a carbon source comprising CO and/or CO2 is provided to the aqueous medium in a continuous gas flow. Even more in particular, the continuous gas flow comprises synthesis gas and oxygen. In one example, both gases are part of the same flow/stream. In another example, each gas is a separate flow/stream provided to the aqueous medium. These gases may be divided for example using separate nozzles that open up into the aqueous medium, frits, membranes within the pipe supplying the gas into the aqueous medium and the like. The oxygen may be free oxygen. According to any aspect of the present invention, ‘a reaction mixture comprising free oxygen’ refers to the reaction mixture comprising elemental oxygen in the form of O₂. The O₂ may be dissolved oxygen in the reaction mixture. In particular, the dissolved oxygen may be in the concentration of 5ppm (0.000005% vol; 5×10⁻⁶). A skilled person may be capable of using any method known in the art to measure the concentration of dissolved oxygen. In one example, the dissolved oxygen may be measured by Oxygen Dipping Probes (Type PSt6 from PreSens Precision Sensing GmbH, Regensburg, Germany.

Step (b) of the method according to any aspect of the present invention involves contacting the acetate and/or ethanol from step (a) with a third microorganism capable of converting the acetate and/or ethanol to the amino acid. In particular, the third microorganism may be genetically modified to comprise increased expression relative to the wild type cell of homoserine acetyl transferase (E₁), aspartokinase (E₂) and homoserine dehydrogenase (E₃); and at least one enzyme selected from a group consisting of phosphoenolpyruvate carboxylase (E₄), aspartate aminotransferase (E₅) and aspartate semi-aldehyde dehydrogenase (E₆).

According to any aspect of the present invention, the first, second and/or third microorganism may be a genetically modified microorganism. The genetically modified cell or microorganism may be genetically different from the wild type cell or microorganism. The genetic difference between the genetically modified microorganism according to any aspect of the present invention and the wild type microorganism may be in the presence of a complete gene, amino acid, nucleotide etc. in the genetically modified microorganism that may be absent in the wild type microorganism. In one example, the genetically modified microorganism according to any aspect of the present invention may comprise enzymes that enable the microorganism to produce at least one amino acid. The wild type microorganism relative to the genetically modified microorganism according to any aspect of the present invention may have none or no detectable activity of the enzymes that enable the genetically modified microorganism to produce at least one amino acid. As used herein, the term ‘genetically modified microorganism’ may be used interchangeably with the term ‘genetically modified cell’. The genetic modification according to any aspect of the present invention may be carried out on the cell of the microorganism.

The phrase “wild type” as used herein in conjunction with a cell or microorganism may denote a cell with a genome make-up that is in a form as seen naturally in the wild. The term may be applicable for both the whole cell and for individual genes. The term “wild type” therefore does not include such cells or such genes where the gene sequences have been altered at least partially by man using recombinant methods. The term ‘wild type’ may also include cells which have been genetically modified in other aspects (i.e. with regard to one or more genes) but not in relation to the genes of interest. The term “wild type” therefore does not include such cells where the gene sequences of the specific genes of interest have been altered at least partially by man using recombinant methods. Therefore, in one example, a wild type cell with respect to enzyme E₁ may refer to a cell that has the natural/non-altered expression of the enzyme E₁ in the cell. The wild type cell with respect to enzyme E₂, E₃, E₄, E₅, E₆ etc. may be interpreted the same way and may refer to a cell that has the natural/non-altered expression of the enzyme E₂, E₃, E₄, E₅, E₆, etc. respectively in the cell.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least twice, especially at least 10 times, at least 100 times, at least 1000 times or at least 10000 times more amino acid than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (amino acid) in the nutrient medium.

The term “second microorganism” or “third microorganism”, refers to a microorganism that is different from “the first microorganism” according to any aspect of the present invention.

In particular, the third microorganism may be any eukaryotic or prokaryotic microorganism that may be genetically modified. More in particular, the third microorganism may be a strain selected from the group consisting of Escherichia sp., Erwinia sp., Serratia sp., Providencia sp., Corynebacteria sp., Pseudomonas sp., Leptospira sp., Salmonellar sp., Brevibacteria sp., Hypomononas sp., Chromobacterium sp., Norcardia sp., fungi and yeasts. Even more in particular, the third microorganism may be selected from Escherichia sp. or Corynebacteria sp., For example, the third microorganism according to any aspect of the present invention may be Escherichia coli or Corynebacterium glutamicum.

The phrase ‘the genetically modified cell has an increased activity, in comparison with its wild type, in enzymes’ as used herein refers to the activity of the respective enzyme that is increased by a factor of at least 2, in particular of at least 10, more in particular of at least 100, yet more in particular of at least 1000 and even more in particular of at least 10000.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures. Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. Similarly, a decreased activity of an enzyme refers to decreased intracellular activity. In one example, the increased expression of an enzyme according to any aspect of the present invention may be 5, 10, 15, 20, 25, 30, 25, 40, 45, 50, 55, 60, 65, 70, 75, 80, 85, 90, 95 or 100% more relative to the expression of the enzyme in the wild type cell. Similarly, the decreased expression of an enzyme according to any aspect of the present invention may be 5, 10, 15, 20, 25, 30, 25, 40, 45, 50, 55, 60, 65, 70, 75, 80, 85, 90, 95 or 100% less relative to the expression of the enzyme in the wild type cell.

According to any aspect of the present invention, the third microorganism may be genetically modified to comprise increased expression relative to the wild type cell of homoserine acetyl transferase (metX) (E₁), aspartokinase (E₂) and homoserine dehydrogenase (thrA) (E₃); and at least one enzyme selected from a group consisting of phosphoenolpyruvate carboxylase (ppc) (E₄), aspartate aminotransferase (aspC) (E₅) and aspartate semi-aldehyde dehydrogenase (asd) (E₆). A skilled person may easily be able to determine the means of measuring the activity of these enzymes by any method known in the art. For example, the activity of homoserine acetyl transferase (E₁) (EC 2.3.1.31) may be a measurement of the reaction:

acetyl-CoA+L-homoserine=CoA+O-acetyl-L-homoserine

Protocols that can be used to measure the activity of enzyme El may at least be found in the following articles: Yamagata, S Journal of Bacteriology, (1987), 169: 3458-63; Park S D et al., Molecules and Cells, (1998), 8(3):286-94; Savin, M. A., et al., Journal of Bacteriology, (1972), 111(2): 547-56, and Lawrence, David A. Journal of Bacteriology, (1972), 109(1): 8-11.

The activity of aspartokinase (aspartate kinase) (E2) (EC 2.7.2.4) may be a measurement of the reaction:

ATP+L-aspartate=ADP+4-phospho-L-aspartate

Protocols that can be used to measure the activity of enzyme E₂ may at least be found in the following articles: Bewas D. K., et al, The Journal of Biological Chemistry, (1968), 243 (13): 3655-3660; and Starnes, W. L., et al., Biochemistry, (1972), 11 (5), pages 677-687.

The activity of homoserine dehydrogenase (E3) (EC 1.1.1.3) may be a measurement of the reaction:

L-homoserine+NAD(P)+=L-aspartate 4-semialdehyde+NAD(P)H+H+

Protocols that can be used to measure the activity of enzyme E₃ may at least be found in the following articles: Starnes, W. L., et al., Biochemistry, (1972), 11 (5), pages 677-687 and Chassagnole, C., et al., Biochemical Journal (2001), 356: 415-423.

The activity of phosphoenolpyruvate carboxylase (E₄) (EC 4.1.1.32) may be a measurement of the reaction:

phosphate+oxaloacetate=phosphoenolpyruvate+HCO3—

Protocols that can be used to measure the activity of enzyme E₄ may at least be found in the following articles: EP 2495317 A1 and Koffas, M. A. G., et al., Applied and Environmental Microbiology, (2002), 68 (11), 5422-5428.

The activity of aspartate aminotransferase (E₆) (EC 2.6.1.1) may be a measurement of the reaction:

L-aspartate+2-oxoglutarate=oxaloacetate+L-glutamate

Protocols that can be used to measure the activity of enzyme E₅ may at least be found on the website as of 8 Jul. 2015, http://www.worthington-biochem.com/cgot/assay.html. The activity of enzyme E₅ may also be measured based on the following article: Muriana F J, et al., Biochemical Journal, (1991), 278 (1), 149-154.

The activity of aspartate semi-aldehyde dehydrogenase (E₆) (EC 1.2.1.11) may be a measurement of the reaction:

L-aspartate 4-semialdehyde+phosphate+NADP+=L-4-aspartyl phosphate+NADPH+H+

Protocols that can be used to measure the activity of enzyme E₆ may at least be found in the following articles: Chassagnole, C., et al., Biochemical Journal (2001), 356: 415-423 and Hegeman, G. D., et al., Methods in Enzymology (1970); 17, Part A, 708-713.

The metX gene may code for homoserine O-acetyltransferase that may be responsible for the first step of the methionine bionsynthesis pathway, which aids in the production of O-acetyl homoserine. E₁ used according to any aspect of the present invention may be from a variety of microorganisms. Examples of the microorganisms from which a gene coding for homoserine O-acetyltransferase may be obtained from include Corynebacterium sp., Leptospira sp., Deinococcus sp., Pseudomonas sp., and/or Mycobacterium sp.. In particular, the homoserine O-acetyltransferase may be encoded by a gene originating from a strain selected from the group consisting of Corynebacterium glutamicum, Leptospira meyeri, Deinococcus radiodurans, Pseudomonas aeruginosa and Mycobacterium smegmatis. More in particular, the homoserine O-acetyltransferase may be from Corynebacterium glutamicum. In one example, the third microorganism used according to any aspect of the present invention may be the strain MH2O-22B hom_fbr disclosed in Sahm et al., (Sahm et al. Ann N Y Acad Sci. 1996 May 15;782:25-39).

Increased expression relative to the wild type cell of aspartokinase (E₂) and/or homoserine dehydrogenase (E₃) may also be included in the third microorganism. Generally thrA refers to a gene encoding a peptide having the activity of aspartokinase and homoserine dehydrogenase. In one example, the aspartokinase and homoserine dehydrogenase may be encoded by a gene of Uniprot database Accession No: AP_000666. In one example, the third microorganism used according to any aspect of the present invention may be the strain MH2O-22B hom_fbr disclosed in Sahm et al., 1996. In another example, the third microorganism may be that disclosed in EP2290051.

In one example, there is provided a method of producing at least acetyl-homoserine from a carbon source in aerobic conditions, the method comprising:

(a) step of producing ethanol and/or acetate from the carbon source in aerobic conditions, comprising
(i) contacting a reaction mixture comprising
   a first acetogenic microorganism in an exponential growth phase;
   free oxygen; and
   a second acetogenic microorganism in a stationary phase

wherein the first and second acetogenic microorganism is capable of converting the carbon source to the acetate and/or ethanol; and

(b) step of contacting the acetate and/or ethanol from step (a) with a third microorganism capable of converting the acetate and/or ethanol to the acetyl-homoserine.

In particular, the third microorganism capable of converting the acetate and/or ethanol to the acetyl-homoserine may be genetically modified to comprise increased expression relative to the wild type cell of homoserine acetyl transferase (E1), aspartokinase (E2) and homoserine dehydrogenase (E3); and at least one enzyme selected from a group consisting of phosphoenolpyruvate carboxylase (E4), aspartate aminotransferase (E5) and aspartate semi-aldehyde dehydrogenase (E6).

The culture medium to be used must be suitable for the requirements of the particular strains. Descriptions of culture media for various microorganisms are given in “Manual of Methods for General Bacteriology”.

All percentages (%) are, unless otherwise specified, mass percent.

With respect to the source of substrates comprising carbon dioxide and/or carbon monoxide, a skilled person would understand that many possible sources for the provision of CO and/or CO₂ as a carbon source exist. It can be seen that in practice, as the carbon source of the present invention any gas or any gas mixture can be used which is able to supply the microorganisms with sufficient amounts of carbon, so that acetate and/or ethanol, may be formed from the source of CO and/or CO₂.

Generally for the cell of the present invention the carbon source comprises at least 50% by weight, at least 70% by weight, particularly at least 90% by weight of CO₂ and/or CO, wherein the percentages by weight-% relate to all carbon sources that are available to the cell according to any aspect of the present invention. The carbon material source may be provided.

Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the mixed culture of the present invention.

According to any aspect of the present invention, the carbon source may be synthesis gas. Synthesis gas can for example be produced as a by-product of coal gasification. Accordingly, the microorganism according to any aspect of the present invention may be capable of converting a substance which is a waste product into a valuable resource.

In another example, synthesis gas may be a by-product of gasification of widely available, low-cost agricultural raw materials for use with the mixed culture of the present invention to produce substituted and unsubstituted organic compounds.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Syngas may also be a product of electrolysis of CO₂. A skilled person would understand the suitable conditions to carry out electrolysis of CO₂ to produce syngas comprising CO in a desired amount. Usually, a portion of the synthesis gas obtained from the gasification process is first processed in order to optimize product yields, and to avoid formation of tar. Cracking of the undesired tar and CO in the synthesis gas may be carried out using lime and/or dolomite. These processes are described in detail in for example, Reed, 1981.

Mixtures of sources can be used as a carbon source.

According to any aspect of the present invention, a reducing agent, for example hydrogen may be supplied together with the carbon source. In particular, this hydrogen may be supplied when the C and/or CO₂ is supplied and/or used. In one example, the hydrogen gas is part of the synthesis gas present according to any aspect of the present invention. In another example, where the hydrogen gas in the synthesis gas is insufficient for the method of the present invention, additional hydrogen gas may be supplied.

A skilled person would understand the other conditions necessary to carry out the method according to any aspect of the present invention. In particular, the conditions in the container (e.g. fermenter) may be varied depending on the first, second and third microorganisms used. The varying of the conditions to be suitable for the optimal functioning of the microorganisms is within the knowledge of a skilled person.

In one example, the method according to any aspect of the present invention may be carried out in an aqueous medium with a pH between 5 and 8, 5.5 and 7. The pressure may be between 1 and 10 bar.

The term “contacting”, as used herein, means bringing about direct contact between the cell according to any aspect of the present invention and the medium comprising the carbon source in step (a) and/or the direct contact between the third microorganism and the acetate and/or ethanol from step (a) in step (b). For example, the cell, and the medium comprising the carbon source may be in different compartments in step (a). In particular, the carbon source may be in a gaseous state and added to the medium comprising the cells according to any aspect of the present invention.

In particular, the aqueous medium may comprise the cells and a carbon source comprising CO and/or CO₂ for step (a) to be carried out. More in particular, the carbon source comprising CO and/or CO₂ is provided to the aqueous medium comprising the cells in a continuous gas flow. Even more in particular, the continuous gas flow comprises synthesis gas. These gases may be supplied for example using nozzles that open up into the aqueous medium, frits, membranes within the pipe supplying the gas into the aqueous medium and the like.

The overall efficiency, alcohol productivity and/or overall carbon capture of the method of the present invention may be dependent on the stoichiometry of the CO2, CO, and H₂ in the continuous gas flow. The continuous gas flows applied may be of composition CO2 and H₂. In particular, in the continuous gas flow, concentration range of CO2 may be about 10-50%, in particular 3% by weight and H₂ would be within 44% to 84%, in particular, 64 to 66.04% by weight. In another example, the continuous gas flow can also comprise inert gases like Nz, up to a Nz concentration of 50% by weight.

The term ‘about’ as used herein refers to a variation within 20 percent. In particular, the term “about” as used herein refers to +/−20%, more in particular, +/−10%, even more in particular, +/−5% of a given measurement or value.

A skilled person would understand that it may be necessary to monitor the composition and flow rates of the streams. Control of the composition of the stream can be achieved by varying the proportions of the constituent streams to achieve a target or desirable composition. The composition and flow rate of the stream can be monitored by any means known in the art. In one example, the system is adapted to continuously monitor the flow rates and compositions of the streams and combine them to produce a single blended substrate stream in a continuous gas flow of optimal composition, and means for passing the optimised substrate stream to the cell according to any aspect of the present invention.

Microorganisms which convert CO2 and/or CO to acetate and/or ethanol, in particular acetate, as well as appropriate procedures and process conditions for carrying out this metabolic reaction is well known in the art. Such processes are, for example described in WO9800558, WO2000014052 and WO2010115054.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep inventive cells, for example LB medium in the case of E. coli, ATCC1754-Medium may be used in the case of C. ljungdahlii. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium. The cells are incubated with the carbon source sufficiently long enough to produce the desired product, 3HB and variants thereof. For example for at least 1, 2, 4, 5, 10 or 20 hours. The temperature chosen must be such that the cells according to any aspect of the present invention remains catalytically competent and/or metabolically active, for example 10 to 42° C., preferably 30 to 40° C., in particular, 32 to 38° C. in case the cell is a C. ljungdahlii cell.

Step (a) and step (b) may be carried out in two different containers. In one example, step (a) may be carried out in fermenter 1 wherein the first and second microorganisms come in contact with the carbon source to produce acetate and/or ethanol. Ethanol and/or acetate may then be brought into contact with a third microorganism in fermenter 2 to produce at least one amino acid. The amino acid and/or the desired amino acid may then be extracted and the remaining carbon substrate fed back into fermenter 1. A cycle may be created wherein the acetate and/or ethanol produced in fermenter 1 may be regularly fed into fermenter 2, the acetate and/or ethanol in fermenter 2 may be converted to at least one amino acid and the unreacted carbon source in fermenter 2 fed back into fermenter 1. Oxygen may be added into fermenter 2 to enable the third microorganism to convert acetate to at least one amino acid. When the remaining carbon source is cycled back from fermenter 2 to fermenter 1, consequently small amounts of oxygen and amino acids may enter fermenter 1. The presence of these small amounts of oxygen and amino acids may still allow for the first and second microorganisms to carry out their activity of converting carbon to acetate and/or ethanol.

In another example, the media is being recycled between fermenters 1 and 2. Therefore, the amino acid produced in fermenter 2 may be fed back into fermenter 1 to accumulate the amino acid produced according to any aspect of the present invention in the fermenters. In the process of recycling the media, oxygen from fermenter 2 and the amino acids produced in fermenter 2 are consequently reintroduced into fermenter 1. As can be seen in the examples, the amino acids may not be metabolised by the microorganisms in fermenter 1. Accordingly, the amino acids may accumulate in the media within the two fermenters. Also, the microorganisms in fermenter 1 may be able to continue producing acetate and ethanol in the presence of the oxygen recycled from fermenter 2 into fermenter 1. The accumulated amino acids may then be extract by means known in the art.

Means of extracting amino acids according to any aspect of the present invention may include an aqueous two-phase system for example comprising polyethylene glycol, capillary electrolysis, chromatography and the like. In one example, when chromatography is used as the means of extraction, ion exchange columns may be used. In another example, amino acids may be precipitated using pH shifts. A skilled person may easily identify the most suitable means of extracting amino acids by simple trial and error.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Formation of Acetic Acid from Syngas in the Presence of Lysine and Homoserine Conserving the Amino Acids using Clostridium ljungdahlii

For the biotransformation of hydrogen and carbon dioxide to acetic acid the homoacetogenic bacterium Clostridium ljungdahlii was cultivated on synthesis gas in the presence of lysine and homoserine conserving the amino acids. All cultivation steps were carried out under anaerobic conditions in pressure-resistant glass bottles that can be closed air-tight with a butyl rubber stopper.

For the preculture of C. ljungdahlii 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; _{0.2} mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochlorid and 400 mg/L Na₂S×9 H₂O were inoculated with 5 mL of a frozen cryo stock. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 69 h till OD₆₀₀ₙₘ>0.4. The gas was discharged into the medium through a sparger with a pore size of 10 pm, which was mounted in the center of the reactors. Then the cell suspension was centrifuged, washed with fresh carbonate buffer and centrifuged again. For the production phase, as many washed cells from the preculture of C. ljungdahlii as necessary for an OD₆₀₀ₙₘ of 0.2 were added to 100 ml carbonate buffer (pH 6.7, 1.4 g/L KOH, 0.4 g/L L-cysteine-hydrochlorid, 0.5 g/L L-homoserine, 0.5 g/L L-lysine, aerated for 30 min with a premixed gas with 67% H₂ and 33% CO₂). The cultivation was carried out in a 500 mL pressure-resistant glass bottle at 37° C., 150 rpm in an open water bath shaker for 165 h and was aerated to an overpressure of 0.8 bar with a premixed gas with 67% H₂, 33% CO₂ one time a day. During cultivation several 5 mL samples were taken to determinate OD₆₀₀ₙₘ, pH and product formation. The determination of the product concentrations was performed by semiquantitative ¹H-NMR spectroscopy. As an internal quantification standard sodium trimethylsilylpropionate (T(M)SP) was used.

During the production phase the concentration of acetate increased from 0.0 g/L to 2.5 g/L and the concentration of ethanol increased from 0.0 g/L to 0.2 g/L. During this time the concentrations of L-homoserine and L-lysine remained constant at 0.5 g/L each.

### Example 2

### Formation of Homoserine from Acetic Acid with Corynebacterium glutamicum

For the biotransformation of acetate to L-homoserine the genetically modified Corynebacterium glutamicum MH20-22B hom_fbr strain was used. The strain C. glutamicum MH20-22B is a chemical mutant which is described by Schrumpf et al. (Appl Microbiol Biotechnol (1992) 37:566-571) and expresses a feedback-resistant aspartate kinase. The strain was modified by Sahm et al. (Ann N Y Acad Sci. 1996 May 15;782:25-39) to strain C. glutamicum MH20-22B hom_fbr, which expresses additionally a feedback-resistant homoserine dehydrogenase.

C. glutamicum MH20-22B hom_fbr was cultivated on BHI agar plates (7.8 g/L brain extract, 2.0 g/L glucose, 2.0 g/L Na₂HPO₄, 9.7 g/L heart extract, 10.0 g/L pepton, 5.0 g/L NaCI, 15.0 g/L agar, pH 7.4) at 30° C.

For the preculture 50 ml of LB medium (5 g/L yeast extract, 10 g/L tryptone, 10 g/L NaCI, pH 7.0, with additional 17.95 g/L potassium acetate) in a 500 ml shaking flask were inoculated with a full loop of cells from a fresh incubated agar plate and cultivated at 30° C. and 120 rpm for 23 h to an OD₆₀₀ₙₘ>3.0. Then the cell suspension was centrifuged, washed with fresh CGF1 medium and centrifuged again.

For the main culture 30 ml of fresh CGF1medium (pH=7.2, 2 g/L (NH₄)₂SO₄, 1 g/L KH₂PO₄, 1 g/L K₂HPO₄, 10 mg/L FeSO₄×7 H₂O, 7.6 mg/L MnSO₄×1 H₂O, 0.246 g/L MgSO₄×7 H₂O, 10.8 g/L ammonium acetate, 0.1 g/L L-leucine) in a 500 ml shaking flask were inoculated with centrifuged and washed cells from the preculture to an OD₆₀₀ₙₘ of 1.0. This culture was incubated in an open water bath shaker at 30° C. and 120 rpm for 120 h. After 46 h of cultivation 7.82 g/L ammonium acetate were added. At the start and during the culturing period, samples were taken. These were tested for optical density, pH and the different analytes (tested by NMR).

During the cultivation phase the concentration of L-homoserine increased from 0 to 497 mg/L, for L-lysine from 0 to 685 mg/L, for L-threonine from 0 to 747 mg/L, for L-isoleucine from 0 to 201 mg/L, for L-glycine from 0 to 243 mg/L, for L-glutamate from 0 to 394 mg/L, for L-valine from 0 to 9 mg/L and for L-alanine from 0 to 45 mg/L. During this time acetate was consumed completely to 0 g/L.

### Example 3

### Formation of Lysine and Other Amino Acids from Acetic Acid with Corynebacterium glutamicum

For the biotransformation of acetate to L-lysine the strain Corynebacterium glutamicum MH20-22B was used. The strain is a chemical mutant which is described by Schrumpf et al. (Appl Microbiol Biotechnol (1992) 37:566-571) and expresses a feedback-resistant aspartate kinase. C. glutamicum MH20-22B was cultivated on BHI agar plates (7.8 g/L brain extract, 2.0 g/L glucose, 2.0 g/L Na₂HPO₄, 9.7 g/L heart extract, 10.0 g/L pepton, 5.0 g/L NaCl, 15.0 g/L agar, pH 7.4) at 30° C.

For the preculture 50 ml of LB medium (5 g/L yeast extract, 10 g/L tryptone, 10 g/L NaCI, pH 7.0, with additional 17.95 g/L potassium acetate) in a 500 ml shaking flask were inoculated with a full loop of cells from a fresh incubated agar plate and cultivated at 30° C. and 120 rpm for 23 h to an OD₆₀₀ₙₘ>4.0. Then the cell suspension was centrifuged, washed with fresh CGF1 medium and centrifuged again.

For the main culture 30 ml of fresh CGF1medium (pH=7.2, 2 g/L (NH₄)₂SO₄, 1 g/L KH₂PO₄, 1 g/L K₂HPO₄, 10 mg/L FeSO₄×7 H₂O, 7.6 mg/L MnSO₄×1 H₂O, 0.246 g/L MgSO₄×7 H₂O, 10.8 g/L ammonium acetate, 0.1 g/L L-leucine) in a 500 ml shaking flask were inoculated with centrifuged and washed cells from the preculture to an OD₆₀₀ₙₘ of 1.0. This culture was incubated in an open water bath shaker at 30° C. and 120 rpm for 120 h. After 46 h of cultivation 7.82 g/L ammonium acetate were added. At the start and during the culturing period, samples were taken. These were tested for optical density, pH and the different analytes (tested by NMR).

During the cultivation phase the concentration of L-lysine increased from 0 to 1,4 g/L, for L-glutamate from 0 to 1,3 g/L, for L-homoserine from 0 to 161 mg/L, for L-threonine from 0 to 140 mg/L, for L-isoleucine from 0 to 48 mg/L, for L-glycine from 0 to 88 mg/L, for L-valine from 0 to 7 mg/L and for L-alanine from 0 to 37 mg/L. During this time acetate was consumed completely to 0 g/L.

### Example 4

### Culture of Clostridium ljungdahlii in Log Phase in the Presence of Synthesis Gas Comprising CO₂ and 0.15% Oxygen

C. ljungdahlii was fed H₂ and CO₂ out of the feed-through gas phase and formed acetate and ethanol. For the cultivation, pressure-resistant glass bottle that can be sealed airtight with a butyl rubber stopper were used. All cultivation steps, where C. ljungdahlii cells were involved were carried out under anaerobic conditions.

For cell culture of C. ljungdahlii 5 mL Cryoculture was cultured anaerobically in 500 ml of medium (ATCC1754 medium: pH 6.0; 20 g/L MES; 1 g/L yeast extract, 0.8 g/L NaCl, 1 g/L NH4Cl, 0.1 g/L KCl, 0.1 g/L KH₂PO₄, 0.2 g/L MgSO₄×7 H₂O; 0.02 g/L CaCl₂×2 H₂O; 20 mg/L nitrilotriacetic acid 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B₁₂; 50 μg/L p-aminobenzoate; 50 μg/L lipoic acid, approximately 67.5 mg/L NaOH) with about 400 mg/L L-cysteine hydrochloride and 400 mg/L Na₂S×9 H₂O. Cultivation was carried chemolithoautotrophically in a flameproof 1 L glass bottle with a premixed gas mixture composed of 67% H₂, 33% CO2 in an open water bath shaker at 37° C., 100 rpm and a fumigation of 3 L/h for 72 h. The gas entry into the medium was carried out by a filter with a pore size of 10 microns, and was mounted in the middle of the reactor, ata gassing tube. The cells were centrifuged, washed with 10 ml ATCC medium and centrifuged again.

For the main culture many washed cells from the growth culture of C. ljungdahlii were transferred into 500 mL of ATCC medium with about 400 mg/L L-cysteine hydrochloride and grown to an OD₆₀₀ of 0.1. Cultivation was carried out in a pressure-resistant 1 L glass bottle with a premixed gas mixture composed of 66.85% H₂, 33% CO₂, 0.15% O₂ in an open water bath shaker at 37 ° C., 150 rpm and with aeration of 1 L/h for 45 h. The gas entry into the medium was carried out by a filter with a pore size of 10 microns, which was placed in the middle of the reactors. When sampling each 5 ml sample was removed for determination of OD₆₀₀ nm, pH and the product range. The determination of the product concentration was performed by semi-quantitative 1 H-NMR spectroscopy. As an internal quantification standard sodium trimethylsilylpropionate served (T (M) SP).

There was significant cell growth shown during the cultivation period, evidenced by an increase in OD₆₀₀ nm of 0.10 to 0.54, corresponding to a growth rate μ=0.037 h⁻¹. The concentration of acetate increased at the same time from 9.6 mg/L to 3,304 mg/L and the concentration of ethanol from 2.2 mg/L to 399 mg/L.

### Example 5

### Culture of Clostridium ljungdahlii in Log Phase in the Presence of Synthesis Gas Comprising CO and 0.1% Oxygen

C. ljungdahlii was autotrophically cultivated in complex medium with synthesis gas, consisting of CO, H₂ and CO₂ in the presence of oxygen in order to produce acetate and ethanol.

A complex medium was used consisting of 1 g/L NH4Cl, 0.1 g/L KCl, 0.2 g/L MgSO₄×7 H₂O, 0.8 g/L NaCl, 0.1 g/L KH₂PO₄, 20 mg/L CaCl₂×2 H₂O, 20 g/L MES, 1 g/L yeast extract, 0.4 g/L L-cysteine-HCl, 0.4 g/L Na₂S×9 H₂O, 20 mg/L nitrilotriacetic acid, 10 mg/L MnSO₄×H₂O, 8 mg/L (NH₄)₂Fe(SO₄)₂×6 H₂O, 2 mg/L CoCl₂×6 H₂O, 2 mg/L ZnSO₄×7 H₂O, 0.2 mg/L CuCl₂×2 H₂O, 0.2 mg/L Na₂MoO₄×2 H₂O, 0.2 mg/L NiCl₂×6 H₂O, 0.2 mg/L Na₂SeO₄, 0.2 mg/L Na₂WO₄×2 H₂O, 20 μg/L biotin, 20 μg/L folic acid, 100 μg/L pyridoxine-HCl, 50 μg/L thiamine-HCl×H₂O, 50 μg/L riboflavin, 50 μg/L nicotinic acid, 50 μg/L Ca-pantothenoic acid, 1 μg/L vitamine B12, 50 μg/L p-aminobenzoic acid, 50 μg/L lipoic acid.

The autotrophic cultivation was performed in 500 mL medium in a 1 L serum bottle that was continuously gassed with synthesis gas consisting of 67.7% CO, 3.5% H₂ and 15.6% CO₂ at a rate of 3.6 L/h. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 120 min⁻¹. pH was not controlled.

At the beginning of the experiment, C. ljungdahlii was inoculated with an OD₆₀₀ of 0.1 with autotrophically grown cells on H₂/CO₂. Therefore, C. ljungdahlii was grown in complex medium under continuous gassing with synthesis gas consisting of 67% H₂ and 33% CO₂ at a rate of 3 L/h in 1 L serum bottles with 500 mL complex medium. Above described medium was also used for this cultivation. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 150 min⁻¹. The cells were harvested in the logarithmic phase with an OD₆₀₀ of 0.49 and a pH of 5.03 by anaerobic centrifugation (4500 min⁻¹, 4300 g, 20° C., 10 min). The supernatant was discarded and the pellet was resuspended in 10 mL of above described medium. This cell suspension was then used to inoculate the cultivation experiment. Gas phase concentration of carbon monoxide was measured sampling of the gas phase and offline analysis by a gas chromatograph GC 6890N of Agilent Technologies Inc. with an thermal conductivity detector. Gas phase concentration of oxygen was measured by a trace oxygen dipping probe from PreSens Precision Sensing GmbH. Oxygen concentration was measured by fluorescence quenching, whereas the degree of quenching correlates to the partial pressure of oxygen in the gas phase. Oxygen measurement indicated a concentration of 0.1% vol of O₂ in the used synthesis gas.

During the experiment samples of 5 mL were taken for the determination of OD₆₀₀, pH and product concentrations. The latter were determined by quantitative ¹H-NMR-spectroscopy.

After inoculation of C. ljungdahlii, cells began to grow with a growth rate μ of 0,062 h⁻¹ and continuously produced acetate up to a concentration of 6.2 g/L after 94.5 hours. Concomitant to the production of acetate, ethanol was produced in a lower rate compared to the production of acetate up to a concentration of 1 g/L after 94.5 hours.

TABLE 1
results of example 4

NMR-analytics

| Process | | | Acetate, | Ethanol, |
|---|---|---|---|---|
| time, h | pH | OD600 | mg/L | mg/L |
| 0.0 | 6.15 | 0.10 | 18 | n.d. |
| 18.0 | 5.97 | 0.69 | 973 | 97 |
| 42.5 | 5.20 | 1.50 | | |
| 66.0 | 4.67 | 1.95 | 5368 | 966 |
| 94.5 | 4.54 | 1.77 | 6187 | 1070 |

(n.d. = not detected)

### Example 6

### Growth and acetate production by Clostridium ljungdahlii on synthesis gas with 2% oxygen

For the biotransformation of hydrogen and carbon dioxide to acetic acid the homoacetogenic bacterium Clostridium ljungdahlii was cultivated on synthesis gas with oxygen. All cultivation steps were carried out under anaerobic conditions in pressure-resistant glass bottles that can be closed airtight with a butyl rubber stopper.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochlorid and 400 mg/L Na₂S×9 H₂O were inoculated with 5 mL of a frozen cryo stock of C. ljungdahlii. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 72 h. The gas was discharged into the medium through a sparger with a pore size of 10 μm, which was mounted in the center of the reactors. Culturing was carried out with no pH control.

After the precultivation, the cell suspension was centrifuged (10 min, 4200 rpm) and the pellet was washed with 10 ml medium and centrifuged again. For the main culture, as many washed cells from the preculture as necessary for an OD₆₀₀ₙₘ of 0.1 were transferred in 200 mL medium with additional 400 mg/L L-cysteine-hydrochlorid. The chemolithoautotrophic cultivation was carried out in a 250 mL pressure-resistant glass bottles at 37° C., 150 rpm and a ventilation rate of 1 L/h with a premixed gas with 65% H₂, 33% CO2, 2%O₂ in an open water bath shaker for 47 h. The gas was discharged into the medium through a sparger with a pore size of 10 μm, which was mounted in the center of the reactors. Culturing was carried out with no pH control. During cultivation several 5 mL samples were taken to determinate OD600nm, pH and product formation. The determination of the product concentrations was performed by semiquantitative 1H-NMR spectroscopy. As an internal quantification standard sodium trimethylsilylpropionate (T(M)SP) was used. Also the dissolved oxygen in the cultivation medium was measured online by oxygen dipping probes (PSt6 with Oxy4Trace, Presens, Germany).

During the cultivation period cell growth was observed by an increase of the OD₆₀₀ₙₘ from 0.11 to 0.32, which correlates with a growth rate of p=0.022 h The concentration of acetate increased from 8 mg/L to 91 mg/L, an increase of the ethanol concentration was not observed. Over the cultivation period the dissolved oxygen concentration varied between 0.06 and 0.15 mg/L. In a similar technical setting with the same parameters (medium composition, volume, bottle, gas, ventilation rate, temperature, shaking frequency), but without cells in the medium, a dissolved oxygen concentration of 0.50 mg/L was measured.

### Example 7

### Growth and Acetate Production by Clostridium ljungdahlii on Synthesis Gas with 0.15% Oxygen

For the biotransformation of hydrogen and carbon dioxide to acetic acid the homoacetogenic bacterium Clostridium ljungdahlii was cultivated on synthesis gas with oxygen. All cultivation steps were carried out under anaerobic conditions in pressure-resistant glass bottles that can be closed airtight with a butyl rubber stopper.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H2O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochlorid and 400 mg/L Na₂S×9 H₂O were inoculated with 5 mL of a frozen cryo stock of C. ljungdahlii. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 72 h. The gas was discharged into the medium through a sparger with a pore size of 10 μm, which was mounted in the center of the reactors. Culturing was carried out with no pH control.

After the precultivation, the cell suspension was centrifuged (10 min, 4200 rpm) and the pellet was washed with 10 ml medium and centrifuged again. For the main culture, as many washed cells from the preculture as necessary for an OD₆₀₀ₙₘ of 0.1 were transferred in 200 mL medium with additional 400 mg/L L-cysteine-hydrochlorid. The chemolithoautotrophic cultivation was carried out in a 250 mL pressure-resistant glass bottles at 37° C., 150 rpm and a ventilation rate of 1 L/h with a premixed gas with 66.85% H₂, 33% CO₂, 0.15% O₂ in an open water bath shaker for 47 h. The gas was discharged into the medium through a sparger with a pore size of 10 μm, which was mounted in the center of the reactors. Culturing was carried out with no pH control. During cultivation several 5 mL samples were taken to determinate OD₆₀₀ₙₘ, pH and product formation. The determination of the product concentrations was performed by semiquantitative 1H-NMR spectroscopy. As an internal quantification standard sodium trimethylsilylpropionate (T(M)SP) was used. Also the dissolved oxygen in the cultivation medium was measured online by oxygen dipping probes (PSt6 with Oxy4Trace, Presens, Germany).

During the cultivation period cell growth was observed by an increase of the OD₆₀₀nm from 0.10 to 0.45, which correlates with a growth rate of p =0.032 h The concentration of acetate increased from 7 mg/L to 2347 mg/L and the concentration of ethanol increased from 2 mg/L to 319 mg/L. Over the whole cultivation period the dissolved oxygen concentration was 0.00 mg/L. In a similar technical setting with the same parameters (medium composition, volume, bottle, gas, ventilation rate, temperature, shaking frequency), but without cells in the medium, a dissolved oxygen concentration of 0.03 mg/L was measured.

### Example 8

### Formation of Acetyl-homoserine from Acetic Acid with Corynebacterium glutamicum

For the biotransformation of acetate to O-acetyl-L-homoserine the genetically modified strain Corynebacterium glutamicum MH20-22B hom_fbr pECXC99E-{Ptrc}[metX_Cg] was used. The strain C. glutamicum MH20-22B is a chemical mutant which is described by Schrumpf et al. (Appl Microbiol Biotechnol (1992) 37:566-571) and expresses a feedback-resistant aspartate kinase. The strain was modified by Sahm et al. (Ann N Y Acad Sci. 1996 May 15;782:25-39) to strain C.

glutamicum MH20-22B hom_fbr, which expresses additionally a feedback-resistant homoserine dehydrogenase. Additionally, this strain was transformed with the plasmid pECXC99E-{Ptrc}[metX_Cg], which encodes for a homoserine O-acetyltransferase (MetX) from Corynebacterium glutamicum ATCC13032.

Corynebacterium glutamicum MH20-22B hom_fbr pECXC99E-{Ptrc}[metX_Cg] was cultivated on BHI agar plates (7.8 g/L brain extract, 2.0 g/L glucose, 2.0 g/L Na₂HPO₄, 9.7 g/L heart extract, 10.0 g/L pepton, 5.0 g/L NaCl, 15.0 g/L agar, pH 7.4, with additional 7.5 mg/L chloramphenicol) for 72 h at 30° C.

For the preculture 4×50 ml of LB medium (5 g/L yeast extract, 10 g/L tryptone, 10 g/L NaCl, pH 7.0, with additional 17.95 g/L potassium acetate and 7.5 mg/L chloramphenicol) in a 500 ml shaking flask were inoculated with a full loop of cells from a fresh incubated agar plate and cultivated at 30° C. and 120 rpm for 22 h to an OD₆₀₀ₙₘ>3.0. Then the cell suspension was centrifuged, washed with fresh CGF1 medium and centrifuged again.

For the main culture 200 ml of fresh CGFlmedium (pH=7.2, 2 g/L (NH₄)₂SO₄, 1 g/L KH₂PO₄, 1 g/L K₂HPO₄, 10 mg/L FeSO₄×7 H₂O, 7.6 mg/L MnSO₄×1 H₂O, 0.246 g/L MgSO₄×7 H₂O, 10.8 g/L ammonium acetate, 0.1 g/L L-leucine, with additional 7.5 mg/L chloramphenicol) in a 500 mL glass bottle were inoculated with centrifuged and washed cells from the preculture to an OD₆₀₀ₙₘ of 1.0. The cultivation was carried out in a pressure-resistant glass bottle that can be closed airtight with a butyl rubber stopper. The culture was incubated in an open water bath shaker at 30° C., 120 rpm and a ventilation rate of 4 L/h with synthetic air (79.5% N₂, 20.5% O₂) for 137 h. The air was discharged into the medium through a sparger with a pore size of 10 μm, which was mounted in the center of the reactors. The pH was held at 7.2 by automatic addition of 25% acetic acid. After 21 h, 1 mM IPTG was added for induction. At the start and during the culturing period, samples were taken. These were tested for optical density, pH and the different analytes by NMR. During the cultivation phase the concentration of O-acetyl-L-homoserine increased from 0 to 230 mg/L, for L-lysine from 0 to 2100 mg/L, for L-threonine from 0 to 410 mg/L, for L-isoleucine from 0 to 19 mg/L, for L-glycine from 0 to 630 mg/L, for L-glutamate from 0 to 150 mg/L, for L-homoserine from 0 to 340 mg/L and for L-alanine from 0 to 140 mg/L. During this time 35.2 g/L acetate were consumed.

1. A method of producing at least one amino acid from a carbon source in aerobic conditions, the method comprising:
(a) step of producing ethanol and/or acetate from the carbon source in aerobic conditions, comprising
(i) contacting a reaction mixture comprising
a first acetogenic microorganism in an exponential growth phase;
free oxygen; and
a second acetogenic microorganism in a stationary phase
wherein the first and second acetogenic microorganism is capable of converting the carbon source to the acetate and/or ethanol; and
(b) step of contacting the acetate and/or ethanol from step (a) with a third microorganism capable of converting the acetate and/or ethanol to at least one amino acid.

2. The method according to claim 1, wherein the amino acid is selected from the group consisting of L-glycine, L-glutamate, L-lysine, L-homoserine, L-isoleucine L-threonine, acetyl-homoserine and L-alanine.

3. The method according to claim 1, wherein the amino acid is L-homoserine and/or acetyl-homoserine.

4. The method according to claim 3, wherein the third microorganism is genetically modified to comprise increased expression relative to the wild type cell of homoserine acetyl transferase (E₁), aspartokinase (E₂) and homoserine dehydrogenase (E₃); and at least one enzyme selected from a group consisting of phosphoenolpyruvate carboxylase (E₄), aspartate aminotransferase (E₅) and aspartate semi-aldehyde dehydrogenase (E₆).

5. The method according to claim 1, wherein the first and second microorganism is selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446, Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950), Butyribacterium methylotrophicum (DSM 3468), Clostridium aceticum (DSM 1496), Clostridium autoethanogenun (DSM 10061, DSM 19630 and DSM 23693), Clostridium carboxidivorans (DSM 15243), Clostridium coskatii (A TCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii O-52 (ATCC 55989), Clostridium mayombei (DSM 6539), Clostridium methoxybenzovorans (DSM 12182), Clostridium neopropionicum sp, Clostridium ragsdalei (DSM 15248), Clostridium scatologenes (DSM 757), Clostridium species ATCC 29797, Desulfotomaculum kuznetsovii (DSM 6115), Desulfotomaculum thermobezoicurn subsp. thermosyntrophicum (DSM 14055), Eubacterium limosum (DSM 20543), Methanosarcina acetivorans C2A (DSM 2834), Moorefla sp. HUC22-1, Mooreila thermoacetica (DSM 521), Moorella thermoautotrophica (DSM 1974), Oxobacter pfennigii (DSM 322), Sporomusa aerivorans (DSM 13326), Sporomusa ovate (DSM 2662), Sporomusa silvacetica (DSM 10669), Sporomusa sphaeroides (DSM 2875), Sporomusa termitida (DSM 4440) and Thermoanaerobacter kivui (DSM 2030).

6. The method according to claim 1, wherein the first acetogenic microorganism in the exponential growth phase has a growth rate of 0.01 to 2 h′¹.

7. The method according to claim 1, wherein the first acetogenic microorganism in the exponential growth phase has an OD₆₀₀ of 0.01 to 2.

8. The method according to claim 1, wherein the aerobic conditions is a result of oxygen being at a concentration of 0.000005-1% volume in the gas phase.

9. The method according to claim 1, wherein the third microorganism is a strain selected from the group consisting of Escherichia sp., Erwinia sp., Serratia sp., Providencia sp., Corynebacteria sp., Pseudomonas sp., Leptospira sp., Salmonellar sp., Brevibacteria sp., Hypomononas sp., Chromobacterium sp., Norcardia sp., fungi and yeasts.

10. The method according to claim 1, wherein the third microorganism is a strain selected from the group consisting of Corynebacteria sp and Escherichia sp.

11. The method according to claim 4, wherein the homoserine acetyl transferase (E₁) is derived from a microorganism selected from the group consisting of Escherichia sp., Corynebacterium sp., Leplospira sp., Deinococcus sp., Pseudomonas sp. and Mycobacterium sp.

12.) The method according to claim 1, wherein the first and/or second microorganism is Clostridium ljungdahlii and the third microorganism is Corynebacterium glutarnicum.

13. The method according to claim 1, wherein the first and/or second microorganism is Clostridium ljungdahlii and the third microorganism is Escherichia coli.

14. The mixture according to claim 1, wherein the carbon source comprises CO.

15. The method according to claim 1, wherein steps (a) and (b) are carried out in separate fermenters.
